(19) United States
(12) Patent Application Publication
(10) Pub. No.: US 2016/0183835 A1
(43) Pub. Date: Jun. 30, 2016

(54) WEARABLE REMOTE ELECTROPHYSIOLOGICAL MONITORING SYSTEM

(71) Applicant: Nanowear Inc., Brooklyn, NY (US)

(72) Inventor: Vijay K. VARADAN, Fayetteville, AR (US)

(73) Assignee: Nanowear Inc., Brooklyn, NY (US)

(21) Appl. No.: 14/965,213

(22) Filed: Dec. 10, 2015

Related U.S. Application Data
(63) Continuation of application No. 13/449,755, filed on Apr. 18, 2012, now abandoned.

(51) Int. Cl. A61B 5/0408; A61B 7/04; A61B 5/08; A61B 5/00; A61B 5/0205

(52) U.S. Cl. CPC A61B 5/04085; A61B 5/0002; A61B 5/02055; A61B 5/08; A61B 5/6804; A61B 7/04; A61B 2562/0209

(57) ABSTRACT

A wearable remote electrophysiological monitoring system. The system includes a garment having at least one nanostructured, textile-integrated electrode attached thereto; a control module in electrical communication with the at least one nanostructured, textile-integrated sensor, and a remote computing system in communication with the control module.

### BACKGROUND

The present invention relates to a physiological monitoring garment.

Heart related ailments like coronary heart diseases, cardiovascular diseases, and strokes that are caused by clots or hypertension are the predominant causes of mortality in the US among both men and women. However, the number of deaths due to cardiac ailments in women has been consistently higher than in men since as early as 1985. In 2006, mortality due to all cardiac ailments among women was nearly 60% more than that due to all forms of cancer combined. This difference is also imminent in the case of post operative survival among women after major cardiac surgeries like coronary bypass. At age 40 and older, 23 percent of women compared with 18 percent of men die within one year after a heart attack. This statistic has been related to the post-menopausal hormonal changes like the levels of estrogen in the blood. Estrogen has been known to have a prophylactic effect on the formation and growth of arterial plaques and clots, which can stifle the flow of blood through major blood vessels or stop it altogether. However, administration of Estrogen and Progestin has been shown to have minimal effect on the outcome of cardiovascular diseases in post-menopausal women.

Chronic diseases such as asymptomatic myocardial ischemia, a decrease in blood supply to the heart, appear as episodic events that do not leave any diagnostic evidence behind, making them all the more difficult to identify. Detection of Cardiac arrhythmias or irregular beats from continuous electroencephalogram (ECG) recordings is an important metric that physicians use to adjust medication for post myocardial infarction patients.

The major risk factors that have been reported to affect the cardiac health of women are smoking, inactivity, obesity, diabetes mellitus and hormonal changes resulting from menopause. Subtle changes in the cardiac activity manifested as irregular heartbeats, aberrational variations in the body's autonomous regulation of blood pressure and minor transient blockages in flow of blood to the heart, due to such chronic conditions or risk factors lead to fatal cardiac episodes. Thus, the best recourse is to engage in preventive measures involving continuous real-time monitoring to better track these physiological changes. Moreover, techniques like Electrocardiograph (ECG), blood pressure, heart rate variability analysis through time, frequency and wavelet domain analysis techniques have been successful in tracking the above-mentioned subtle changes.

### SUMMARY

To that end, the sensors required to pick up the necessary biological signals and constantly relay the signals need to be seamlessly integrated into everyday clothing such that no additional preparation or mounting of individual sensors is needed. The innovative ‘e-bra’ described here is a foundation garment or a brassiere, designed with a multitude of sensor capabilities for cardiac and pulmonary health monitoring which are integrated into a fabric with improved performance. The end result is an autonomous garment that can collect and transmit vital health signals of the wearer.

The e-bra will also help non-critical users (i.e. those not acutely suffering from a condition such as heart or pulmonary diseases) for monitoring important metrics such as calories burned during a workout, to get an optimum workout by jogging or on a treadmill, and pacing their exercise. For instance, the wearer's heart rate should be at the proper intensity level for an extended period of time. If the heart rate gets too high, the wearer's activity can become counterproductive. If it is too low, the wearer is not getting optimal health benefits. This technology will thus monitor and provide the optimum workout needed for a given individual.

The e-bra system described here is a comfortable and wearable monitor for cardiovascular and pulmonary health for women. It has a basic structure of a foundation garment for woman's bosom that covers all or part of chest, shoulders, arms and upper back. Sensor components include biopotential electrodes like electrocardiogram (ECG) electrodes which are mounted on the garment, photoplethysmography channels which are worn as an arm band, piezoelectric acoustic sensors, temperature sensors, and piezoresistive respiration effort sensors.

This technology also provides additional benefits even if one is not a cardiovascular or pulmonary patient. For example, individuals could use the devices to report beneficial activities (exercising, taking medications, sleeping) and receive incentives from partners (doctors, insurance companies, social networks) with whom they share that information.

Thus, in one embodiment the invention provides a wearable remote electrophysiological monitoring system. The system includes a garment having at least one nanostructured, textile-integrated electrode attached thereto; a control module in electrical communication with the at least one nanostructured, textile-integrated sensor, and a remote computing system in communication with the control module.

### BRIEF DESCRIPTION OF THE DRAWINGS

Embodiments of the invention can become more fully understood from the detailed description given herein below and the accompanying drawings, given by way of illustration only and thus not intended to be limitative of the present invention.

FIG. 1(a) shows lead placement for a twelve-lead ECG with derived limb leads.

FIG. 1(b) illustrates the placement of electrodes on the frontal side of the garment in which the electrodes have been placed according to the medical specifications for the limb leads, precordial leads, chest lead, and ground lead.

FIG. 1(c) illustrates the mounting of an electrode on elastic backing using stitching.

FIG. 2 illustrates the back electrode site and the elastic backings provided in the brassiere platform where the elastic backings facilitate the ECG electrodes maintaining contact with the skin.

FIG. 3 shows the position for the acoustic sensor(s), respiration effort sensor and temperature sensor(s).

FIG. 4 shows the back side of a complete brassiere system, with an extended left arm sleeve that can be detached, with the inset showing a photoplethysmography module.

FIG. 5(a) shows a scanning electron image of gold nanowires such as those used in embodiments of the nanostructure-based electrodes.

FIG. 5(b) shows gold nanostructure-containing electrodes mounted on a standard snap-on button.

FIG. 5(c) shows conductive fabric incorporating a textile electrode which includes nanostructures.

FIG. 6 shows a block diagram of an embodiment of the system.

FIG. 7 shows nanostructures projecting from a fiber.

FIG. 8 shows statistics on cardiac related mortalities in females as compared to females in the United States: 1976-2006.

FIG. 9(a) shows placement of electrodes for ECG lead 2.

FIG. 9(b) shows an ECG waveform with characteristic P wave, QRS complex, and T and U waves.

FIG. 10 shows an e-bra worn by a test subject, the control module, and the smartphone display interface.

FIG. 11(a) shows the electrode positions on the e-bra.

FIG. 11(b) shows data acquired from subject 1.

FIG. 11(c) shows data acquired from subject 2.

FIG. 12 shows R-R interval determination from an ECG.

FIG. 13(a) shows a plot of the RR interval series against beat number.

FIG. 13(b) shows a plot of the AR PSD computed from the RRI series for the standing case.

FIG. 14(a) shows a plot of the RR interval series against beat number.

FIG. 14(b) shows a plot of the AR PSD computed from the RRI series for the standing case.

FIG. 15 shows the sequence of processes and steps followed by the cloud server when an emergency abnormal condition reflected by abnormal health data is detected.

FIG. 16 shows the sequence of processes and steps followed on the mobile device in response to an emergency message sent by the cloud server.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

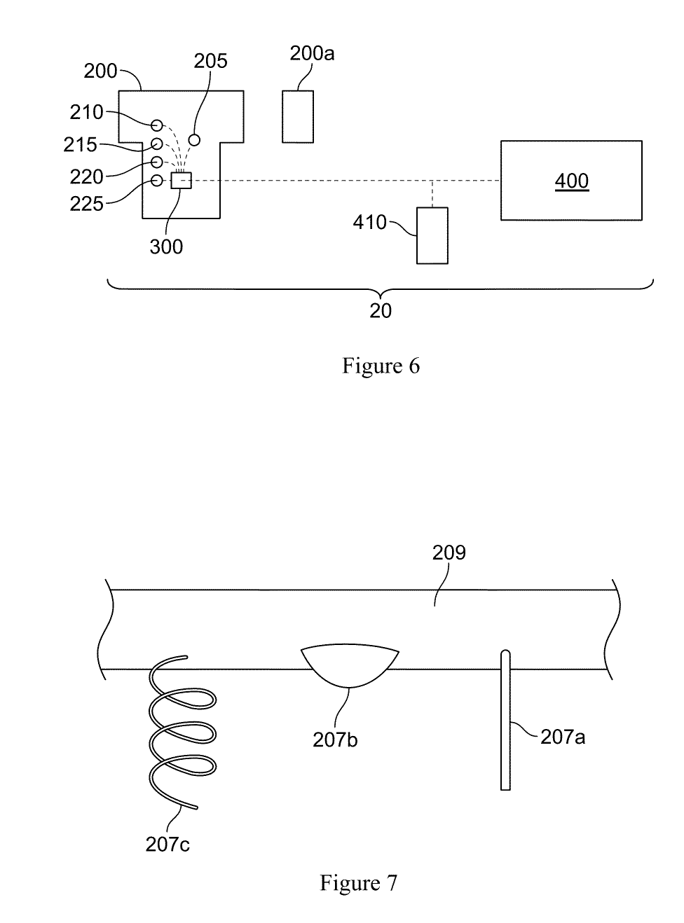

In various embodiments, the invention includes a wearable remote electrophysiological monitoring system 20 (FIG. 6). The system 100 may include a garment 200 having at least one nanostructured, textile-integrated electrode 205 attached thereto, a control module 300 in electrical communication with the at least one nanostructured, textile-integrated electrode 205, and a remote computing system 400 in communication with the control module 300 (FIG. 6). The system may also include a plurality of physiological sensors such as a photoplethysmography sensor 210, an acoustic sensor 215, a temperature sensor 220, and a strain sensor 225 (FIG. 6). The acoustic sensor 215 may be attached to the garment 200 to collect acoustic signals from a heart of a wearer of the garment 200. The temperature sensor 220 may include a resistive temperature detector, a thermistor, and an infrared photodiode detector. The strain sensor 225 may include a piezoresistive respiration effort sensor to monitor breathing of a wearer of the garment 200. The various physiological sensors may be electrically connected to the control module 300 by silver-coated thread. Each group of electrodes or sensors may have an amplifier module associated therewith, for example attached to the garment 200 in the vicinity of the electrodes or sensors or incorporated into the control module 300.

The remote computing system 400 may communicate with the control module 300 using radio-frequency communications, for example using short-range communications such as Bluetooth; a local area network (e.g. wi-fi); satellite; or cellular communications technology. The remote computing system 400 may also communicate with the control module 300 using other forms of communications such as infrared light or microwaves. In some embodiments, the remote computing system 400 may communicate with the control module 300 using a wire-based connection or a combination of wired and wireless modalities.

The nanostructured, textile-integrated electrodes 205 may be made of a hierarchically-organized nanostructure sheet with vertically standing nanowires/filaments. The electrodes 205 are generally incorporated in the fabric of the garment 200 with an elastic backing for concomitant contact with the skin.

The nanostructured, textile-integrated electrodes 205 include nanostructures 207 attached to and projecting from electrically-conductive fibers 209 that may be incorporated into a portion of fabric. The nanostructures 207 may project from the fiber 209 to varying lengths ranging from 0.01-10 micrometers, and in one embodiment project from the fiber 209 less than one micrometer. The portion of fabric may then be attached to or otherwise incorporated into the garment 200 and placed into electrical communication with the control module 300.

The nanostructures 207 projecting from the fiber 209 may have different shapes and form factors and may include one-dimensional nanostructures 207a, two-dimensional nanostructures 207b, and/or three-dimensional nanostructures 207c (FIG. 7). The one-dimensional structures 207a may include approximately linear structures such as wires or tubes. The two-dimensional structures 207b may include shapes such as bumps or bubbles. The three-dimensional structures 207c may include shapes such as helices. The helices are particularly suitable as they have a large surface area available for making contact with a wearer's skin. In some embodiments in which helical structures are employed, a particular handedness of the helices (e.g. left-handed or right-handed) may produce better results such as improved conductivity. The fiber 209 from which the nanostructures 207 project is typically electrically conductive, which may be achieved by using a fiber 209 that is coated with an electrically conductive material (e.g. silver) or by using a fiber 209 that is blended or intertwined with an electrically conductive material (e.g. silver). The nanostructures 207 may be fabricated from a number of different materials such as gold, silver, steel, or textiles. In one embodiment, a piece of fabric having fibers with nanostructures thereon can have a density of between 10,000 and 100,000 nanostructures per square centimeter of fabric.

In various embodiments, the nanostructured, textile-integrated electrodes 205 are used as dry contact sensors, i.e. sensors that do not require a conductive gel or other substance to be used with the electrodes 205 to make electrical contact with the wearer's skin. The base substrate (e.g. fiber 209) is flexible and conductive and can be made of metal or metal-textile blend(s) or metal-polymer blend(s). Possible metals that may be used include gold, silver, titanium, platinum, and steel or a steel alloy, and possible textile fabrics that may be used include nylon, silk, Lycra, spandex, polyester, modified celluloses, and cotton.

In various embodiments, the garment 200 may be a brassiere (also referred to as the e-bra), a vest, a shirt, or other garment worn over the upper body. In general the garment 200 is form-fitting in order to ensure sufficient contact of the various sensors with the skin of the wearer. Generally, the garment 200 conforms to the wearer's body and complies with standard sizing/fitting schemes, including, in the case of an e-bra, standard cup size and strap lengths. Suitable materials for making the garment 200 include nylon, silk, Lycra, spandex, polyester, modified celluloses, cotton, and combinations of these and other materials, and in general the garment 200 is washable. As described herein, the garment 200 includes electrodes/sensors incorporated therein and in some embodiments the garment 200 may be supplemented by one or more armbands 200a (FIG. 6) or other wearable devices for collecting additional data. In various embodiments, the system 20 may be worn underneath the wearer's normal clothing for seamless deployment for monitoring the wearer's cardiovascular health or other health indicators.

In some embodiments, the system 20 includes a plurality of nanostructured, textile-integrated electrodes 205 arranged on the garment to collect an electrocardiogram (ECG) signal from a wearer of the garment 200 (FIG. 1), where the electrodes 205 are located on the garment 200 so as to capture heart activity from different perspectives or positions. Since the electrodes 205 in certain embodiments are textile-based, they can be more readily integrated into the fabric of the garment 200 (e.g. an e-bra).

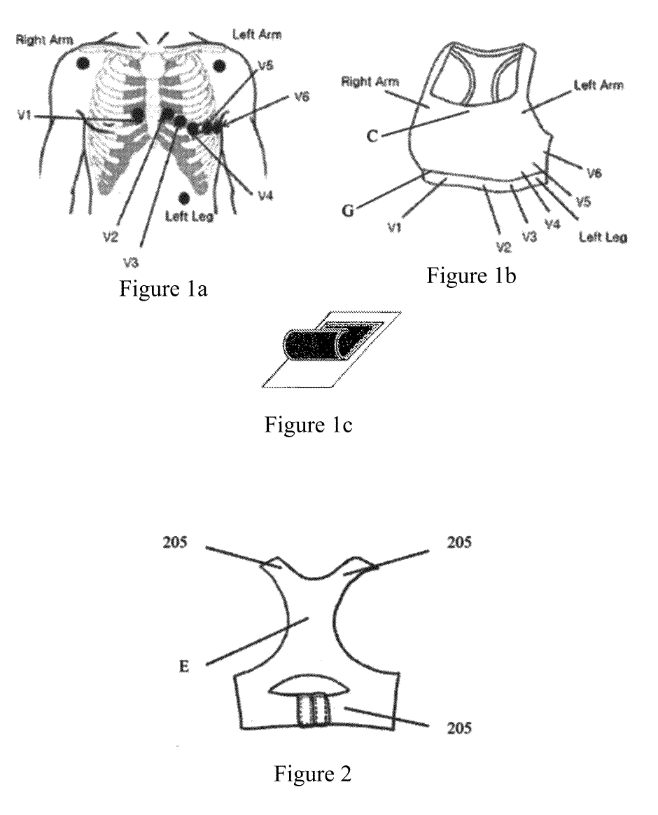

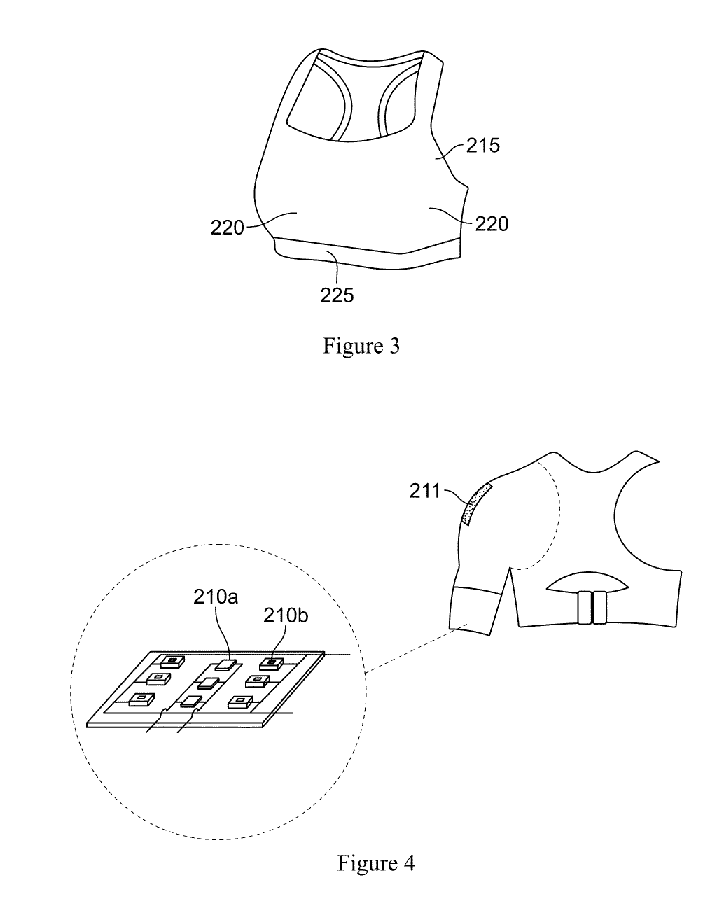

Although there can be variations in the arrangement of electrodes for measuring an electrocardiogram, the positions used in the embodiment depicted in FIGS. 1(a)-1(c) are medically classified as (but not limited to): limb leads: Right Arm, Left Arm, and Left Leg; precordial leads V1-V6; chest lead C; ground G; and experimental lead E at the back (shown in FIG. 2). In one embodiment, the electrodes 205 have conductive fiber-based connections, without using conventional wires, which enable the electrodes to send signals to an on board amplification and transmission system (e.g. which may be integrated into the control module 300).

Plethysmography measurements can be obtained from impedance measurements (as opposed to optical-based photoplethysmography measurements disclosed herein) in conjunction with ECG recording. This provides information regarding pulse transit time from ventricular discharge to the passage of the pulse at the brachial artery site, the brachial artery being located in the upper arm. The pulse transit time bears a correlation with the compliance of the brachial artery; therefore, it can be correlated to the blood pressure in the artery, thus accomplishing a unique non-invasive blood pressure measurement in real time on a continuous basis without the need for an inflatable cuff.

The system 20 may also include a plurality of photoplethysmography sensors 210 or channels, which may be integrated into the garment 200 or coupled to an armband 200a to be worn by the user (FIG. 6). In one embodiment, the photoplethysmography (PPG) channels use combinations of light emitting diodes (LED) 210a and photo detectors (PD) 210b (FIG. 4, inset) that are mounted on the garment 200 (particularly if the garment includes sleeves) and/or an armband 200a, where the armband 200a may be made of a material such as nylon, cotton, Lycra, spandex, neoprene, or other elastomeric fabric or film. The wavelengths of light that are used are generally biocompatible red and infrared. The origin of the observed PPG signals may be due to absorption of the light that is emitted by the LED 210a or may be the reflection of light from the LED 210a by blood.

As with impedance-based plethysmography measurements, photoplethysmography measurements can be used to detect pulse waves in the brachial artery. The LEDs 210a may be arranged in a serial connection and the photo detectors 210b arranged in a parallel connection. The LED-PD combinations include two LEDs 210a flanking one PD 210b (FIG. 4, inset) at separations that constitute a solid geometric angle for optimum detection of the reflected or transmitted light from the deep-seated brachial artery. The combination is designated as one channel that is mounted in the transverse sense to the left brachial artery axis (inwards of the left arm). More than one such channel is used to scan the brachial artery. Such a configuration gives a stronger signal, one that is more tolerant to variations in the placement position of the arm band 200a or sleeve of the garment 200. As discussed above, the use of an armband 200a may be an addition to the system 20 for enhancing monitoring capabilities. In some embodiments in which the garment 200 includes sleeves, the photoplethysmography sensors 210 may be attached directly to the garment 200, in particular to the sleeves.

In those embodiments employing acoustic sensors, the acoustic sensors 215 may be based on a hydrophone pad design. The acoustic sensors 215 may be mounted on the garment 200 (e.g. e-bra) in a position that is suitable for detecting sounds being produced by activity of the heart and/or breathing of the wearer. The signals, recorded through these acoustic sensor 215 systems, are important for diagnosing medical conditions like heart murmur, heart valve activity, respiratory blockages, and subsonic (less than 20 Hertz) and ultrasonic (greater than 20 kilohertz) vibrations of diagnostic value. Piezo-resistive textile-based or textile-integrable strain sensors 225 may be mounted on the garment for detection of thoracic distention towards monitoring the respiration effort and respiration cycle.

In some embodiments, one or more temperature sensors 220 may be mounted on the garment 200. Temperature sensors 220 may be based on resistive temperature detectors, thermistors, or infrared photodiode detectors. As with other electrodes and sensors described herein, the temperature sensors 220 may have conductive fabric- or thread-based connections, i.e. without traditional wires, that enable them to send signals to an onboard amplification and transmission system (e.g. which may be integrated into the control module 300).

In various embodiments, the garment 200 is made of the same material as the textile base for the ECG electrodes. In those embodiments in which the garment 200 includes straps or other connectors, ECG or other electrodes 205 may be placed so as to coincide with the adjustable elastic backings of the straps or other connectors to serve dual purposes, while preserving the overall functionality of the garment 200 (FIG. 2). The connections from the ECG electrodes (FIG. 1(b)) and photoplethysmography device (FIG. 4, inset) are drawn out using fabric-based electrodes made with the same assortment of materials described above. In one embodiment, a garment 200 with a non-standard extended left arm sleeve is provided for accommodating the photoplethysmography band (FIG. 4, inset) and an amplifier-transmitter module with power source 211. The conductive fabric or thread for the conductive fabric- or thread-based connections, which can be made with the same assortment of materials described above, can be stitched on the garment in the form of connective lines that relay the signal from sensors to an onboard amplification-transmission module on a flexible board (e.g. which may be integrated into the control module 300) for seamless integration into the garment 200. The connection scheme can also be optical, which involves enmeshed optical fibers. The gauge of the connective lines is generally a function of the electrical and/or optical ratings of the sensor systems. In various embodiments, the control module 300 can use wireless communication with a remote computing system 400 for data logging and post processing. Given the importance of uninterrupted heart monitoring, the amplifier modules associated with the ECG electrodes of the garment 200 may be equipped to connect to a wired data-logging setup. For example, the amplification circuitry in the amplification modules may include ancillary access points for connecting the respective signal channels to a standard data-logging interface with provisions to one of either a display or a data transmission.

The control module 300 and the remote computing system 400, among other components, are based on standard computer systems having a microprocessor, memory and data storage, input and output, and wired or wireless networking capabilities. The methods and systems described herein may be implemented using one or more such computer systems working in one or more locations to assemble and disseminate data.

The nanostructures 207 of the nanostructured, textile-integrated electrodes 205 (because of their relatively large surface area) are highly sensitive and accurate. Coupled with a low-power microcontroller and Bluetooth module (using one or more of Zigbee, WiFi, and/or other communication protocols as appropriate), the sensor data can be streamed to commercial off-the-shelf cell phones and handheld devices.

In various embodiments the system 20 may include a software application for operation on a smartphone 410 (FIG. 6). The smartphone 410, via the software application, can collect sensor data over Bluetooth or other communications channels and can relay data over 3G, Wi-Fi, WiMax or any outgoing connection using radio-based communications. Using the smartphone 410 and software application, the system 20 does not require any additional custom handheld device for relaying data.

In various embodiments, the software application can provide several additional functions besides basic functions such as data collection and transmission. One possible function is implementation of filtering algorithms on the smartphone 410 to mitigate issues due to motion and other artifacts, rendering cleaner data. In addition, the software application can provide a visualization interface on the smartphone 410 through which users can see salient features of their heart activity such as heart rate. An additional function is that the smartphone 410 software application can tag the data with the location of the wearer of the garment 200. The location (e.g. latitude, longitude) collected is useful for both backend services as well as for the user himself/herself in case of a medical emergency.

In some embodiments, the software application on the smartphone 410 can run machine learning algorithms to perform preliminary anomaly detection. In case of an emergency, it can either alert the wearer and recommend him/her to hospital locations near his/her present location or make an automated call to the wearer's physician or emergency personnel with his/her present location. Thus caregivers can access into vital information anywhere and at any time within the healthcare networks for global level active monitoring. As an indication of the scalability of the system, a Zigbee-based WiFi system is capable of handling 65,000 patients at a given time.

In some embodiments the system 20 may include a Global Positioning System (GPS) module, for example as part of the control module 300. Current location data from the GPS module included in the system 20 can be tagged (e.g. by the control module 300 or by the smartphone 410 software application) to the wearer's data and transferred to a remote (“cloud”) data cluster and in addition can be stored in a secure database (e.g. an SD card can be installed in the control module 300 to save the data). For physician diagnostics a new backend service may be provided in which the doctor can log into a secured database and visually review the past and current sensor data from the garment 200 system 20 (as necessary). If the physician desires, he/she can employ machine learning algorithms (e.g. embedded in the control module 300, the smartphone 410 software application, and/or the remote computing system 400) to detect abnormalities in the data. Further, a VoIP service can be used to make phone calls or send SMS messages to physicians from the wearer. Additionally, the smartphone 410 or other mobile device can send relevant abnormal data in advance to emergency services in the event the wearer receives medical assistance. The smartphone 410 or other mobile device, if equipped with a camera, can prompt the wearer to start a video call. Processes and steps for emergency or other situations are described in FIGS. 6 and 7.

There are a number of uses of the system 20 disclosed herein, including wireless real-time monitoring of heart rate variability (HRV) and/or ECG and detection of asymptomatic myocardial ischemia in diabetic patients. Real-time monitoring using the system 20 also improves quality of life for patients with medical conditions that can elevate chances of asymptomatic (silent) ischemia attack.

Other uses of the system 20 include monitoring the health of the myocardium after administering ischemia-preventive drugs or reperfusion and disease management for patients with chronic coronary heart disease. The sensors, with wireless signal transmission, present a tool that provides real-time ischemia monitoring for patients while maintaining mobility of the patients.

Software (e.g. the smartphone 410 software application) will give the wearer data such as calories burned during workout, exercise, walking, jogging, and other activities. As noted above, monitoring of data from the garment 200 using a smartphone 410 also permits the use of GPS tracking to identify the user's location. The light weight, comfort, and wireless communications capabilities of the system 20 also allow it to be used for monitoring patients with sleep disorders and for continuous monitoring of stroke patients, ECGs, blood pressure, and any vital parameter of the heart functions in Intensive Care Unit (ICU) in the hospital.

### Example

The following non-limiting Example discloses a particular embodiment of the wearable remote electrophysiological monitoring system 20.

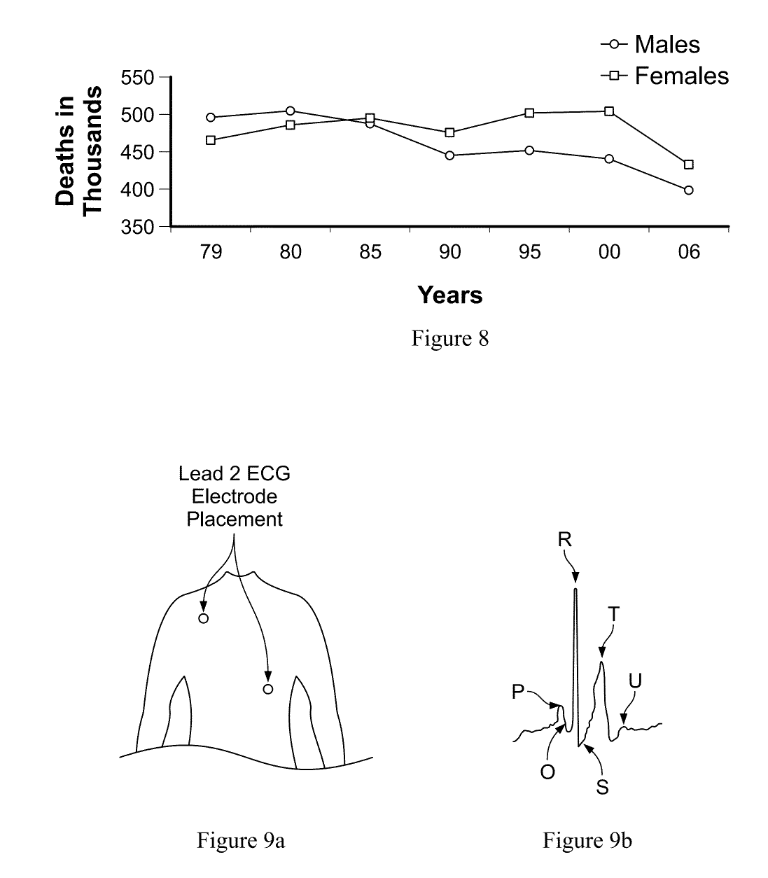

Initial manifestation of most cardiovascular diseases (CVDs) is usually chest pain or angina. Diagnostic tests are then carried out to decide upon a disease management if not a treatment strategy. At this stage the risk factor for women has been shown to be statistically higher than in men. At age 40 and older, 23% of women compared with 18% of men die within one year after a heart attack. Cardiac related mortalities in women have surpassed mortalities due to all cancers by over 60%. As shown by the plot in FIG. 8, CVD related mortality has been consistently higher in women than in men since 1985.

This difference is also imminent in the case of postoperative survival in women after cardiac surgeries. The reasons cited for such a discrepancy range from increased complexity of cardiothoracic surgeries due to the average small frame and consequently small blood vessel size in women, to the lack of a clear understanding of the influence of menopause related hormonal changes on the autonomic nervous control of cardiac activity and vasovagal balance. The female sex hormone, estrogen, has been known to have a prophylactic effect on the formation and growth of arterial plaques and clots that can stifle the flow of blood through major blood vessels or stop it altogether. This observation has been corroborated by studies on heart rate variability (HRV) indicating the increased involvement of vasovagal balance in young women. However, the administration of estrogen or progestin has been shown to have minimal effect on the outcome of cardiovascular diseases in postmenopausal women. These conflicting findings suggest that the best recourse will be to engage in prognostic measures involving continuous real time monitoring to better track and identify any pathophysiological changes.

Chronic diseases cause subtle changes in the cardiac activity, manifested as irregular heartbeats, aberrational variations in the body's autonomous regulation of blood pressure, and minor transient blockages in flow of blood to the heart referred as ischemic attacks. Chronic diseases such as asymptomatic myocardial ischemia, a decrease in blood supply to the heart, manifest as episodic events that do not leave any diagnostic evidence behind beyond 2-3 min after an episode, making them all the more difficult to identify. These attacks can be detected through variations in the ECG waveform characteristics like the ST segment amplitude and width. Women diagnosed with ischemic heart diseases have a higher frequency of symptomatic episodes as compared with men, which results in more hospitalization and associated costs. Moreover, the variation of T wave amplitude and duration, referred to as T wave alternans has been shown to be a predictor of sudden cardiac arrest (SCA) due to ventricular arrhythmias, which is a disease that claims nearly 400,000 individuals every year in the United States. Detection of cardiac arrhythmias or irregular beats from continuous ECG recordings is also an important metric that physicians use for risk stratification and to adjust medication for postmyocardial infarction patients.

Techniques like HRV analysis through time, frequency, and wavelet domain analysis techniques have been successful in tracking autonomic nervous-cardiovascular regulation, which is indicative of chronic diseases as mentioned previously. Thus, various parameters derivable from ECG are of significant prognostic value with regard to CVDs. Sensors that can comprehensively track cardiovascular and pulmonary activity are needed to be able to detect and quantify the electrophysiology of the heart (through ECG), the heart sounds associated with the opening and closing of valves murmur sounds that occur due to inefficient heart valve activity and the activity of the lungs in terms of both the respiratory effort and sounds associated with any blockages or fluid accumulations in the lungs. The full potential of these prognostic tools can be realized only if these sensors can be used. To that end, this paper describes the e-bra, which is used as a platform on which the various sensors for cardiac health monitoring are integrated into the fabric. The end result is an autonomous garment that can collect and transmit vital health signals of the wearer to any desired location in the world through connections to a smartphone and the cellular network or through a Bluetooth enabled PC and the internet. As a first step toward a complete cardiovascular health garment, described herein are means for acquiring ECG signals from a subject using the e-bra, transmitting the data to a smartphone or a Bluetooth enabled PC and perform the above mentioned power spectrum analysis of the HRV.

The use of a smartphone as a base station for receiving data offers the advantage of cellular network connectivity to the Internet and consequently, the availability of cloud computing resources for real time automatic anomaly detection and response to critical emergencies. To address this capability, disclosed herein is a protocol for response to emergencies from both the cloud backend and the smartphone.

The electrocardiogram (ECG) is a simple noninvasive diagnostic test performed to observe any abnormalities in cardiac electrophysiology. The ECG waveform acquired from a derived Lead II electrode placement system is shown in FIG. 9(a), which clearly depicts the classical components of the ECG waveform (FIG. 9(b)). The waveform characteristics of the ECG include P wave, QRS complex, and T and U waves (FIG. 9(b)).

PR interval, QRS duration, ST segment duration, T wave amplitude (referred as T wave alternant), and T wave width are the diagnostically relevant quantities obtained directly from the data. The derived quantities of interest are R peak to R peak interval (for heart rate determination and arrhythmic cardiac activity detection), variability in ST segment duration and amplitude, and power spectrum and wavelet domain analysis of HRV sequences obtained from the RR interval (RRI).

The 12 lead ECG including three augmented limb leads, three limb leads, and six chest leads gives a comprehensive observation of the electrophysiology of the heart from all angles. However, the continuous monitoring of all 12 lead ECG is only required for high risk patients who have already been diagnosed with CVD. Moreover, the use of a full 12 lead system for everyday monitoring can be inconvenient and cumbersome. An alternative five electrode system that gives all the diagnostic information of a 12 lead system can be used for continuous monitoring instead. FIG. 1(a) shows the lead placements for the 12 lead system and FIG. 1(b) shows the similar implementation on the e-bra with textiles or gold nanowire electrodes, where the nanowires may be shaped as one-dimensional (wires) and/or three-dimensional (helices) nanostructures.

The RRI is the time elapsed between the onset of an R peak of the ECG and that of the next and hence signifies the time between two consecutive beats. The variability of this interval is referred to as HRV. It is well known that the human RRI series has three major frequency components: (i) the very low frequency (VLF), (ii) the low frequency (LF), around 0.1 Hz, and (iii) the high frequency (HF), between 0.2 Hz and 0.4 Hz. Consequently, algorithms for extracting the RRI involve an identification of the instances of the R-wave occurrence or of the QRS complex in the ECG data, followed by concatenating the time-differences between successive instances. The RRI series is rich in information about the cardiovascular physiology of a subject.

Although there is some disagreement with respect to the physiological indication of the LF component, most studies consider LF to be an indicator of sympathetic nerve modulation of heart rate. The HF reflects vagal nerve influence on the same. The ratio LF/HF is used as a diagnostic quantity that can reflect the autonomic neuropathy due to chronic diseases such as diabetes. A reduction in this ratio has also been observed in the case of postmyocardial infarction patients. A lower HRV has been shown to be indicative of compromised cardiac health. Thus HRV analysis has been studied as a valuable, noninvasive and easy to implement diagnostic tool. However, it is important to note that factors such as fiducial point selection for HRV calculation, sampling rate, and considerations of data latency are key to obtaining reproducible results. The inclusion of HRV analysis of supine and head-up tilt ECG of a subject acquired through the e-bra validates both the e-bra and the acquisition system as reliable cardiac health monitoring system.

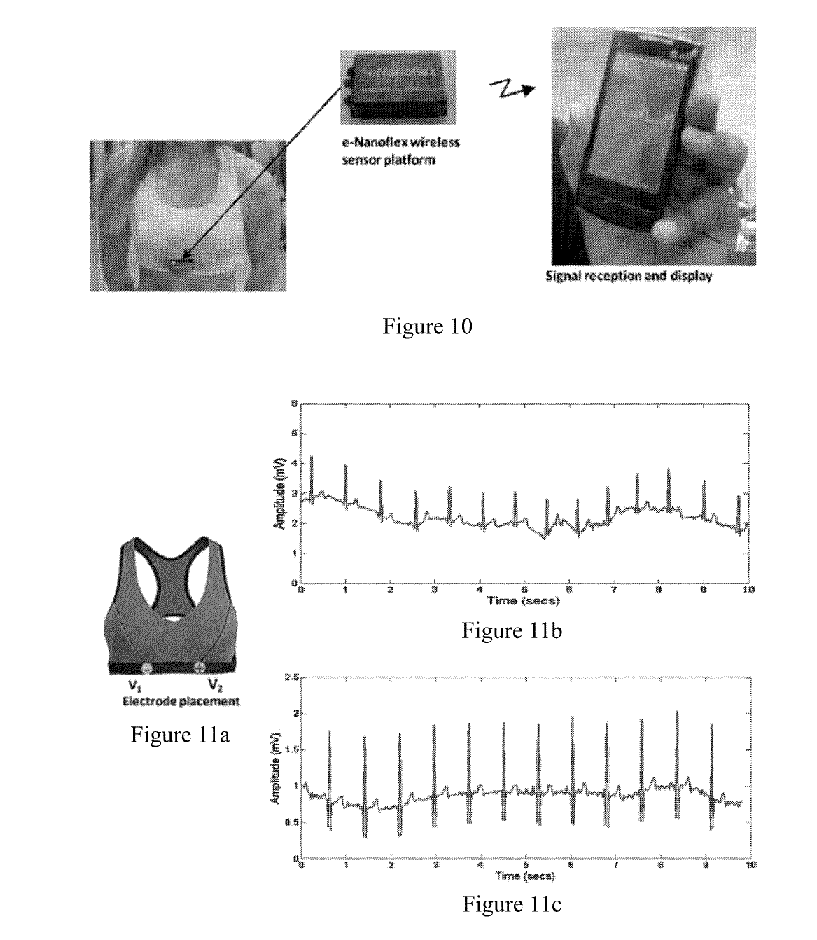

Recent developments in embedded computing and the emergence of smartphones as powerful portable computing devices have made truly pervasive computing a reality. Along with significant computing power, the communication protocols for interdevice communications have also become more reliable and offer high data rates of the order of 3 Mbit/s, e.g. as seen with Bluetooth™ version 2.1. The various sensors disclosed herein are incorporated in the e-bra and the signals from the sensors are brought to the control module through conductive threads, which are made from silver-coated fabric. FIG. 10 shows a picture of the e-bra, the control module used for data acquisition and wireless transmission, and a smartphone display interface for an application that plots the data received from the control module.

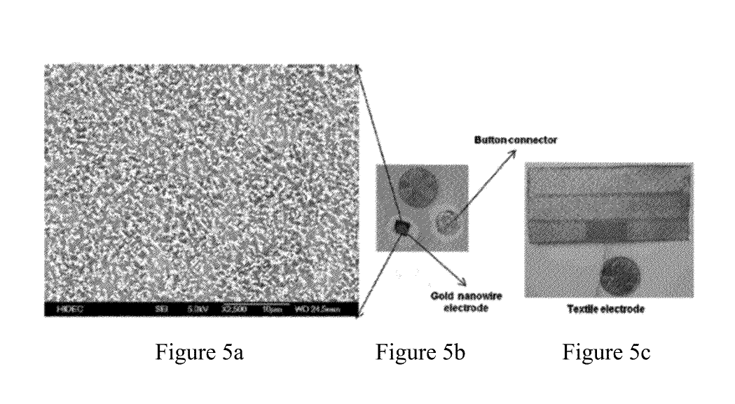

ECG measurements are due to the change in impedance across the heart measured at the level of the skin. It is an information rich signal that is regularly used to diagnose various kinds of cardiac ailments. In this Example, ECG data has been acquired from two subjects with the e-bra, which is a textile based platform with the electrodes mounted on it. Commercially available electrodes for ECG use Ag/AgCl electrodes with a conductive gel that minimizes the impedance between the skin and the electrode. Due to problems of gel drying that results in noise signals and strong adhesives, it causes discomfort when worn for long durations. Dry electrodes offer a much more comfortable and durable alternative. To this end, if a garment needs to be able to pick up good quality ECG and has to be worn every day and throughout the day, it needs to use dry and washable electrodes such as the gold nanowire electrodes or conductive fabric based electrodes that can be easily stitched onto the e-bra. FIGS. 5(a)-5(c) show the gold nanowire electrodes and the conductive fabric electrodes that are used to acquire ECG from the subjects.

In experiments, two electrodes were placed in the positions described as V1 and V2. The difference in potential between these two positions is known to show a distinct and sharp peak in the signal that corresponds to the activation of the left ventricle of the heart, namely, the R peak of the ECG. The left ventricle of the heart pumps blood from the heart to the peripheral arteries and is used as an indicator that corresponds to the completion of one cardiac cycle. The signal also shows Q, S, and T waveforms, where S-T segment is for ventricular repolarization.

A three stage differential amplifier was used with a maximum gain of 65 dB and a 3 dB bandwidth of 0.1-70 Hz. The amplified output signal of the amplifier was digitized by an Atmega 328P microcontroller (Atmel Corporation, San Jose, Calif.) at 200 Hz and transmitted through a Bluetooth module (STMicroelectronics, Geneva, Switzerland).

The signal conditioning algorithms, the R peak detection algorithms, and the HRV analysis were implemented on a PC. Data were acquired from two healthy subjects and the data acquired are plotted in FIGS. 11(b) and 11(c).

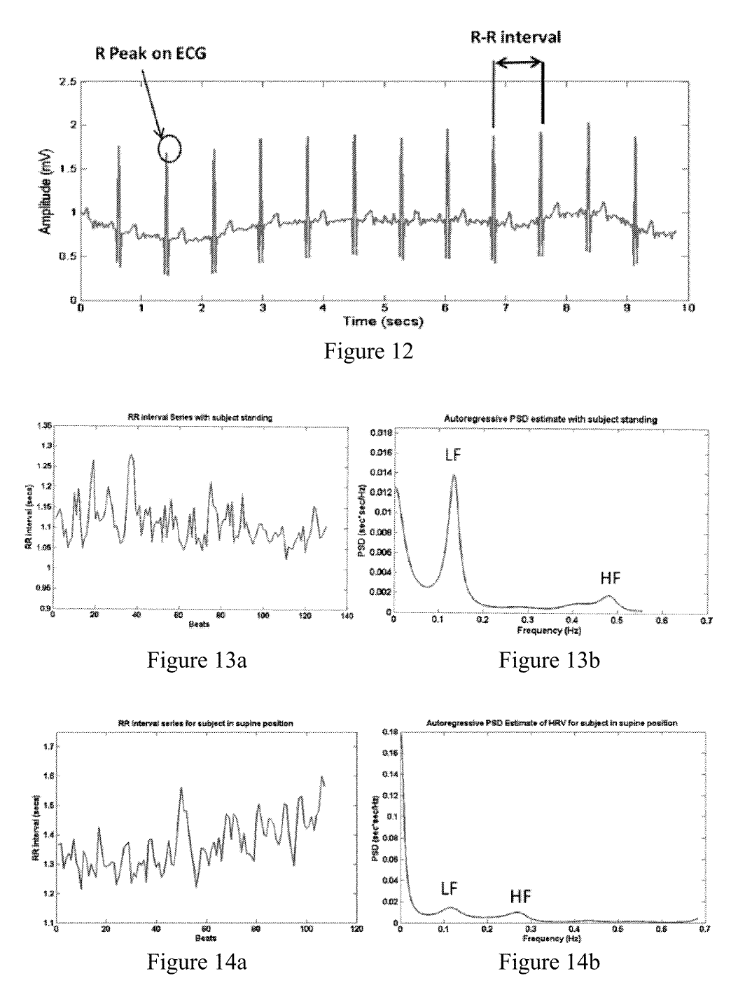

As discussed above, heart rate variability has received great interest as a prognostic and diagnostic tool over the past two decades. Heart rate variability is described as the sequence formed by concatenating the difference in heart rate between consecutive beats. The inverse of this quantity is the difference in the intervals between consecutive R peaks. By detecting the R peaks, the interval between them can be identified and hence obtain the heart rate variability signal against beats, as shown in FIG. 12. A robust R peak detection algorithm was implemented along with a subroutine for calculation of RRI and derivation of HRV.

The autoregressive (AR) power spectrum estimation technique was used to obtain the power spectrum density (PSD) plot with the characteristic LF and HF peaks. The AR PSD is best suited for short data record lengths and performs very well as a frequency estimator for signals with strong sinusoidal components such as the HRV signals. A 150 s record of ECG was collected for a normal healthy female of age 18 in supine position and while standing still. The support of the RR interval signal is beats and the sampling frequency used for AR PSD computation was chosen to be the mean RR interval. FIG. 13(a) shows the plot of the RR interval series plotted against beats and FIG. 13(b) shows the AR PSD computed from the RRI series for the head-up tilt case. FIGS. 14(a) and 14(b) show the same for supine ECG. In the case of head-up tilt, the heart rate was higher as compared with the supine ECG. The classic shift in the power distribution between low-frequency (LF) and high-frequency (HF) components with respect to the total power in each case is evident from the AR PSDs in FIGS. 13(b) and 14(b). Thus, the implementation of an e-bra for cardiac monitoring is shown to be a reliable system for tracking of chronic conditions related to autonomous nervous regulation of cardiac activity.

As mentioned above, complete cardiac monitoring will require real time ECG for the detection of arrhythmic heart beats, ST segment abnormalities associated with ischemic attacks, myocardial infarction, and other waveform characteristics such as PR interval and QRS complex width. These are indicative of the functioning of the atria of the heart and blockages in the cardiac electric conduction pathways, respectively. HRV analysis, on the other hand, provides insight into changes in the regulation of cardiovascular function stemming from chronic diseases such as diabetes and hypertension, which are major risk factors for the occurrence of CVDs.

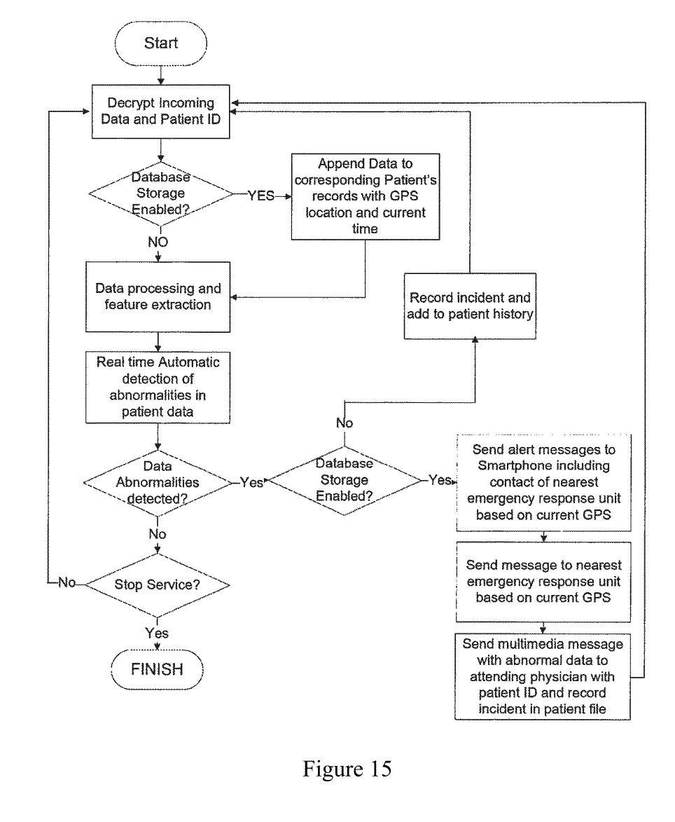

The tracking and assessment of long-term chronic diseases through techniques such as HRV analysis alone does not realize the full potential of the e-bra system. The incorporation of additional intelligence into the system to automate and facilitate quickest possible response to any emergency situation is vital to realize the full potential of this system. Taking this requirement into consideration, the system disclosed herein can harness the computing power of the cloud cluster through the connectivity of the smartphone to the Internet, e.g. through the cellular network. Also proposed is a protocol for the response from a backend server in the event of such an emergency and a concomitant protocol for alerting the wearer through the smartphone. The overall system includes the wearer's Smartphone, a device on the Emergency Medical Service (EMS) vehicle that responds to the emergency, the attending physician's Smartphone, and the wearer's physician. The flow chart in FIG. 15 describes the response at the backend server.

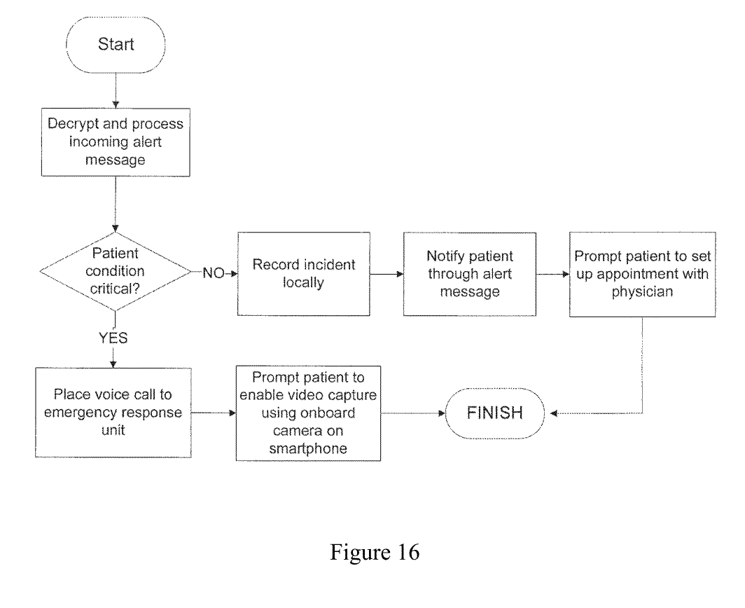

At the Smartphone end, there are a number of standard utilities that can be used in case of an emergency such as the onboard video camera, Voice over Internet Protocol (VoIP) connectivity, and Global Positioning System (GPS). The flow chart in FIG. 16 shows the proposed response protocol at the wearer's Smartphone. The video capture option is included here so that EMS personnel may be able to provide instructions to the wearer as appropriate for the circumstances.

This non-limiting Example describes an embodiment the e-bra platform for the mounting of heart monitoring sensors and the incorporation of wireless communication to this platform. Heart rate variability analysis has been performed based on the data acquired from a subject with the e-bra and it has been shown that the e-bra can be used as a reliable means of assessing chronic cardiac conditions in patients including women. There are a number of advantages of using an automated abnormality detection scheme and a protocol is proposed which can be followed for the response to an emergency from the backend server, the Emergency Medical Service vehicle, the attending physician's phone, and/or the wearer's smartphone.

Thus, the invention provides, among other things, a wearable remote monitoring system. Various features and advantages of the invention are set forth in the following claims.

1. A wearable remote electrophysiological monitoring system, comprising:
a garment having at least one dry contact sensor, the dry contact sensor including at least one nanostructured, textile-integrated electrode attached thereto, the nanostructured, textile-integrated electrode including
a piece of fabric having between 10.000 and 100.000 nanostructures ver square centimeter of fabric, the fabric having electrically conductive fibers, said nanostructures having vertically standing nanowires, the vertically standing nanowires projecting from the fibers to varying lengths of between 0.01 and 10 millimeters, the nanowires arranged to make contact with the wearer's skin;
a control module in electrical communication with the at least one nanostructured, textile-integrated electrode; and
a remote computing system in communication with the control module.

2. The wearable remote electrophysiological monitoring system of claim 1, further comprising a plurality of physiological sensors selected from the group consisting of a photoplethysmography sensor, an acoustic sensor, a temperature sensor, and a strain sensor.

3. The wearable remote electrophysiological monitoring system of claim 2, further comprising a plurality of the nanostructured, textile-integrated electrodes arranged on the garment to collect an electrocardiogram signal from the wearer of the garment.

4. The wearable remote electrophysiological monitoring system of claim 2, wherein the acoustic sensor is attached to the garment to collect acoustic signals from a heart of a wearer of the garment.

5. The wearable remote electrophysiological monitoring system of claim 2, wherein the temperature sensor comprises a resistive temperature detector, a thermistor, and an infrared photodiode detector.

6. The wearable remote electrophysiological monitoring system of claim 2, wherein the strain sensor comprises a piezoresistive respiration effort sensor to monitor breathing of a wearer of the garment.

7. The wearable remote electrophysiological monitoring system of claim 2, further comprising a plurality of photoplethysmography sensors coupled to the garment or an armband.

8. The wearable remote electrophysiological monitoring system of claim 2, wherein the physiological sensors are electrically connected to the control module by silver-coated thread.

9. The wearable remote electrophysiological monitoring system of claim 1, wherein the remote computing system communicates with the control module using radio-frequency communications.

10. (canceled)

11. (canceled)

12. The wearable remote electrophysiological monitoring system of claim 1, wherein each of the plurality of nanostructures project from the fiber less than one micrometer.

13. The wearable remote electrophysiological monitoring system of claim 1, wherein at least one of the plurality of nanostructures comprises a one-dimensional nanostructure.

14. The wearable remote electrophysiological monitoring system of claim 13, wherein the one-dimensional nanostructure comprises a wire or a tube.

15. The wearable remote electrophysiological monitoring system of claim 1, wherein at least one of the plurality of nanostructures comprises a bump.

16. (canceled)

17. (canceled)

18. The wearable remote electrophysiological monitoring system of claim 1, wherein the fiber is electrically conductive.

19. The wearable remote electrophysiological monitoring system of claim 1, wherein the fiber comprises a textile coated with an electrically conductive material.

20. The wearable remote electrophysiological monitoring system of claim 19, wherein the electrically conductive material is silver.

21. The wearable remote electrophysiological monitoring system of claim 1, wherein the fiber comprises a textile intertwined with an electrically conductive material.

22. The wearable remote electrophysiological monitoring system of claim 1, wherein the nanostructures comprise at least one of gold, silver, steel, or textile.

23. The wearable remote electrophysiological monitoring system of claim 1, wherein at least one of the plurality of nanostructures comprises a three-dimensional nanostructure.

24. The wearable remote electrophysiological monitoring system of claim 23, wherein the three-dimensional nanostructure comprises a helical nanostructure.

25. The wearable remote electrophysiological monitoring system of claim 24, wherein the helical structure is a right-handed helical structure
